(19) United States
(12) Patent Application Publication
(10) Pub. No.: US 2022/0288276 A1
(43) Pub. Date: Sep. 15, 2022

(54) ARTIFICIAL ENDOTHELIAL KERATOPLASTY GRAFT AND METHODS OF PREPARATION THEREOF

(71) Applicant: Precise Bio Inc., Winston-Salem, NC (US)

(72) Inventors: Aryeh BATT, Har Hebron (IL); Amos EITAN, Jerusalem (IL); Ariel EISENBACH, Zufim (IL); Yishay HAYARDENI, Greensboro, NC (US); Mehwish KHAN, High Point, NC (US); Michal MARCUS, Nechalim (IL); Kanti NEPAL, Winston Salem, NC (US); Khanh NGUYEN, Winston-Salem, NC (US); Lior SHAV, Glen Rock, NJ (US); Yan WU, Winston-Salem, NC (US)

(21) Appl. No.: 17/198,449

(22) Filed: Mar. 11, 2021

(51) Int. Cl. A61L 27/38; A61F 2/14; A61L 27/24; A61L 27/52; A61L 27/22; A61L 27/36; C08J 3/075; C08J 3/28; C08L 89/06

(52) U.S. Cl. CPC A61L 27/3808; A61F 2/142; A61L 27/24; A61L 27/52; A61L 27/3839; A61L 27/222; A61L 27/3633; C08J 3/075; C08J 3/28; C08L 89/06; A61F 2240/001; A61L 2430/16

(57) ABSTRACT

The present invention discloses an artificial endothelial keratoplasty graft consisting a support layer made of rehydrated crosslinked hydrogel and corneal endothelial cells on top or within said support layer. The invention also discloses a method of manufacturing an artificial endothelial keratoplasty graft, wherein said method consisting of a step of drying support layer material followed by a crosslinking step.

### FIELD OF INVENTION

The present invention generally pertains to an artificial endothelial keratoplasty graft and to methods of its use and preparation.

### BACKGROUND OF THE INVENTION

Corneal blindness is estimated to have effect on the order of 10 million people worldwide. It is further estimated that only about 1% of the people suffering from corneal blindness receive treatment. In part, the low rate of treatment is due to the scarcity of corneal tissue donors. Even in cases in which corneal tissue is available, problems frequently remain, as corneal tissue can easily be damaged during the transplantation procedure, and tends to be less healthy in older donors than in younger ones. Furthermore, the time frame for the donation is quite short, as the tissue should be harvested within several hours of death and transplanted within a few days after harvesting.

The term corneal transplantation is used to describe several different medical procedures, in which different parts of the cornea is being implanted, including: (A) Penetrating Keratoplasty (PK), in which the entire thickness of the cornea is transplanted, (B) Descemet Stripping Endothelial Keratoplasty (DSEK), in which the endothelium membrane, Descemet membrane and part of the posterior corneal stroma are transplanted, and (C) Descemet Membrane Endothelial Keratoplasty (DMEK), in which the endothelium and the Descemet membrane are transplanted, without additional stoma layer.

Among the procedures mentioned above, DSEK is the most common one. In the US, 60% of the total corneal transplantations are DSEK. However, the DMEK procedure has major advantages over it, including lower rejection rate (1% for DMEK compared with 12% of DSEK), higher probability for visual acuity above 0.8 (79% in DMEK compared with 47% in DSEK), and shorter healing period (weeks in DMEK compared with months to year in DSEK).

The main reason that the DMEK procedure is not fully taking over the DSEK is the ease of implantation. In particular, the native DMEK graft tends to break more easily during the procedure, and the endothelial cells in it are sensitive for pressure and shear stresses. Therefore, the DMEK usually requires special implantation techniques and higher experienced surgeons. In addition, since the DMEK is so fragile and sensitive, doctors prefer stiffer graft obtained from donors older than 50 or 60 years. However, the corneal endothelial cells in these grafts are in a lower density and conditions.

Thus, a reliable source for thin yet stiff corneal implants, with healthy and functioning endothelial cells and repeatable conditions, remains unmet need.

### BRIEF DESCRIPTION OF THE DRAWINGS

The various features and advantages of the present invention may be more readily understood with reference to the following detailed description taken in conjunction with the accompanying drawings in which

FIG. 1 schematically illustrating a DMEK-like graft according to one embodiment of the invention;

FIG. 2 schematically illustrating a DMEK-like graft on a designated carrier according to another embodiment of the invention; and

FIG. 3 schematically illustrating a corneal graft orientation in the eye, according to yet another embodiment of the invention.

### SUMMARY OF THE INVENTION

It is thus an object of the current invention to disclose the preparation of PVEK, namely a DMEK-like implant, which may be implanted using the detachable and/or dissolvable carrier device mentioned in the previous file.

The terms “Precise vision endothelial keratoplasty” and PVEK for short, will be interchangeably herein after refer to a set of solutions found in a few cases being equal to, if not better than the DSEK implant, as some the inventors disclosed in U.S. Pat. Appl. No. 62/487,018 “Bioengineered corneal graft and methods of preparation thereof” incorporated herein as a reference. DMEK is configured to be implanted without a carrier and the carrier can be used with native DMEK grafts.

The PVEK-implant of the present invention is very thin, but is still stiff enough to handle with standard ophthalmic tools. Also, native DMEK graft tend to fold due to the contraction of the cells on the Descemet membrane. This makes the implantation much more difficult as the surgeon has to flatten the graft in the eye. Here however, the membrane does not tend to fold and the graft opens more easily after insertion.

The PVEK-grafting by the present technology comprises, inter alia, step(s) of drying out a solution of collagen and/or gelatin on a surface, and crosslinking it with EDC/NHS solution, that bond the collagen and/or gelatin polymers and make it a hydrogel thin film. Since the crosslinking process occurs while the material is not fully wetted, the resulting hydrogel is very thin and with high polymer concentration. These layers can be implanted in a similar way to DMEK, but have an advantage that they do not tend to fold after implantation like DMEK.

The present invention hence discloses, inter alia, an artificial endothelial keratoplasty graft consisting a support layer made of rehydrated crosslinked hydrogel and corneal endothelial cells on top or within said support layer.

The present invention also discloses an artificial endothelial keratoplasty graft as defined above, wherein the rehydrated crosslinked hydrogel is based on crosslinked collagen or collagen methacrylate.

The present invention also discloses an artificial endothelial keratoplasty graft as defined in any of the above, wherein the source of said collagen or collagen methacrylate is human recombinant collagen.

The present invention also discloses an artificial endothelial keratoplasty graft as defined in any of the above, wherein the rehydrated crosslinked hydrogel is based on crosslinked gelatin or gelatin methacrylate.

The present invention also discloses an artificial endothelial keratoplasty graft as defined in any of the above, wherein the total thickness of the graft is between 5 and 50 microns.

The present invention also discloses an artificial endothelial keratoplasty graft as defined in any of the above, wherein the total thickness of the graft is below 25 microns, therefore mimicking native DMEK graft.

The present invention also discloses an artificial endothelial keratoplasty graft as defined in any of the above, wherein the mechanical properties of said support layer allows positioning and flattening of the graft inside the anterior chamber during implantation.

The present invention also discloses an artificial endothelial keratoplasty graft as defined in any of the above, wherein the cells remodeled the said support layers to form corneal endothelium extra-cellular matrix, by a long maturation period or by additional nutritions to the cells.

The present invention further discloses a method of manufacturing an artificial endothelial keratoplasty graft, wherein the method consisting of a step of drying support layer material followed by a crosslinking step.

The present invention also discloses the method as defined above, wherein the support layer material is collagen or collagen methacrylate.

The present invention also discloses the method as defined in any of the above, wherein the source of said collagen or collagen methacrylate is human recombinant collagen.

The present invention also discloses the method as defined in any of the above, wherein the support layer material is gelatin or gelatin methacrylate.

The present invention also discloses the method as defined in any of the above, wherein the crosslinking step involves in introducing EDC and NHS molecules to the material.

The present invention also discloses the method as defined in any of the above, wherein the crosslinking step involves in introducing LAP or 1-[4-(2-Hydroxyethoxy)-phenyl]-2-hydroxy-2-methyl-1-propane-1-one (a commercially available as Irgacure 2959 trademark by Ciba) molecules to the material and applying light on it.

The present invention also discloses the method as defined in any of the above, wherein the drying step is performed in controlled environmental conditions.

The present invention also discloses the method as defined in any of the above, wherein the final thickness of said graft is below 25 microns.

The present invention also discloses the method as defined in any of the above, wherein the mechanical properties of said graft allow pulling and pushing it during implantation, therefore allowing positioning and flattening inside the anterior chamber of the eye.

The present invention also discloses the method as defined in any of the above, wherein the water content in said support material is between 30 and 90 percent.

### DETAILED DESCRIPTION OF THE INVENTION

The artificial Endothelial Keratoplasty Implant and methods of preparation thereof disclosed herein are designed to meet this need, by allowing the production of DMEK-like grafts with healthy and functioning cells and good mechanical properties.

The implant is made of Descemet membrane-like hydrogel support layer, and a layer of corneal endothelial cells (CEC) layer on top. The endothelial layer is made of CEC cells obtained from donors' corneas and proliferated, or by stem-cells differentiation, by methods known in the art.

The support layer is made of a thin collagen or gelatin layer, which was designed to have mechanical properties, transparency and dimensions to improve patients' vision and health condition.

To produce the endothelial keratoplasty, a layer of low concentration collagen solution is spread on a surface and dehydrated. After drying out, a crosslinker solution is introduced to the dried material which form a crosslinked hydrogel in thicknesses between 2 to 50 micrometers.

The excess and un-crosslinked reagents are washed away using PBS and corneal endothelial cells are seeded on top of the hydrogel, forming a two-layer construct, similarly to DMEK cadaver grafts.

In some cases, the corneal graft is implanted onto the posterior cornea using a designated DMEK tools or carrier. In preferred embodiments, prior to the implantation a carrier layer is attached to the corneal graft using biocompatible adhesive material, forming a 4-layers implant: An Endothelial keratoplasty graft made of a collagen/gelatin layer and a cells layer, an adhesive layer, and a carrier. After implantation, the adhesive and the carrier layer are possibly detached from the graft and pulled out or dissolved in the eye.

In other cases, the corneal graft is implanted similarly to cadaver-sourced DMEK grafts, by methods known in the art.

The present invention hence discloses, inter alia, an artificial endothelial keratoplasty graft consisting a support layer made of rehydrated crosslinked hydrogel and corneal endothelial cells on top or within said support layer.

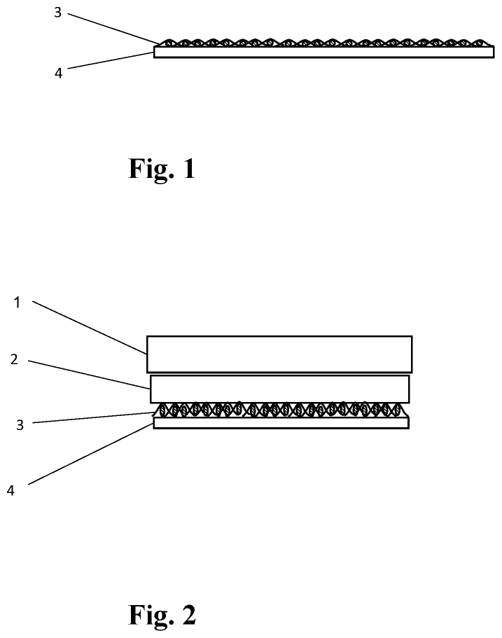

Reference is made to FIG. 1, schematically illustrating a DMEK-like graft according to one embodiment of the invention. Upper layer is corneal endothelial cells (3) and below is a support layer (4).

Reference is now made to FIG. 2, schematically illustrating a DMEK-like graft on a designated carrier according to another embodiment of the invention. upper layer in this illustration is a carrier (1); and below are adhesive (2), Corneal endothelial cells (3) and support layer (4).

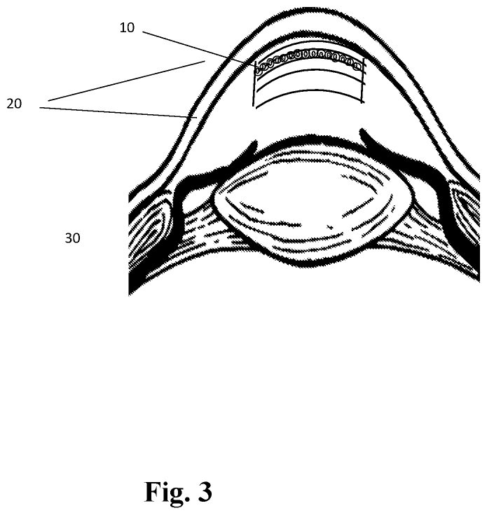

Reference is now made to FIG. 3, schematically illustrating a corneal graft orientation in the eye, according to yet another embodiment of the invention. Upper illustration is a corneal graft with adhesive and carrier layers attached to the posterior cornea (not in scale) (10). Cornea (20) and anterior chamber (30) are also illustrated. Figure is adapted from a currently available public draw https://www.iconspng.com/image/92595/eye-3.

It is according to a few embodiments of the invention wherein the graft comprises two main layers, namely (i) a support (Descemet membrane-like) layer, made of materials selected from a group consisting of collagen, gelatin, collagen methacrylate, gelatin methacrylate and a combination thereof; and (ii), a corneal endothelial cells layer, seeded on top of the support layer.

It is according to a few embodiments of the invention wherein the geometry of the support layer is ranging between about 2 and about 50 microns thick; diameter ranging between about 7.5 to about 9.5 mm.

It is further according to a few other embodiments of the invention wherein the geometry of the cells layer is ranging between about 2 and about 20 microns in thickness, cells density of about 1,500 to about 4,000 cells/mm\hat{ }2; diameter of about 7.5 to about 9.5 mm.

It is according to a few embodiments of the invention wherein the materials of the support layer are selected from a group consisting of Collagen, Collagen methacrylate, Gelatin, Gelatin from about 1 to about 50% w/v, whereas native stroma has 13% collagen.

It is according to a few embodiments of the invention wherein additional materials of the support layer are selected from a group consisting of photo-initiators and crosslinkers: LAP, Irgacure 2959, APS-TEMED, 1-ethyl-3-(3-dimethylaminopropyl) carbodiimide (EDC), N-hydroxysuccinimide (NHS), and a combination thereof.

It is according to a few embodiments of the invention wherein additional materials of the support layer are selected from a group consisting of biocompatible dyes for easy handling such as trypan blue.

It is according to a few embodiments of the invention wherein the materials of the cells layer are selected from a group consisting (i) human corneal endothelial cells from human donors, with or without expansion of the cells by a proliferation step; (ii) human corneal endothelial cells derived from stem cells such as induced pluripotent stem cells (iPSC) or human embryonic stem cells (hESC); (iii) cells own ECM, produced by the cells during the maturation process and any combination thereof.

It is according to a few embodiments of the invention wherein the preparation methods comprise drying step(s). Such a drying steps comprises steps of spreading or molding about 0.1 to about 15% (w/v) materials selected from collagen, collagen methacrylate, gelatin, and gelatin methacrylate on a surface. The spread/molded layer thickness may vary between about 0.1 and about 10 mm. Then, the materials are let to dry at specific range of temperatures, between about 4 and 60 degrees Celsius, and relative humidity levels. Total drying period time is ranging from 1 h to about 10 days. The drying process may be consisted of several different environmental condition steps.

It is according to a few embodiments of the invention wherein the preparation methods comprise crosslinking step(s). After drying, crosslinking the dried sheet by washing it with one or more of the following: EDC, NHS, LAP, Irgacure 2959 or other crosslinker solution, or a mixture of the above. Using photo-initiating light source is possible to induce the process. Crosslinking time between about 1 minute and about 48 hours. Afterwards, washing the crosslinked gel to remove residues of crosslinker and un-crosslinked polymer and immersing it in water/PBS/media. Then, possibly repeating the drying and crosslinking steps multiple times.

The present invention discloses a method of manufacturing an artificial endothelial keratoplasty graft, wherein the method consisting of a step of drying support layer material followed by a crosslinking step.

It is according to a few embodiments of the invention wherein the preparation methods comprise seeding step(s). Seeding corneal endothelial cells on top of the gel. Maturing for about 1 to about 28 days in an incubator.

### Example 1

Spreading 160 μL of 0.5% (w/v) collagen on an area of 10 mm×10 mm, on a flat surface (e.g. Petri dish), to form a layer of about 2 mm thick. Letting the material dry at 4 degrees Celsius, 40% RH, for 48 hours. Adding 2 ml PBS solution with (1 mg/ml) EDC and (0.25 mg/ml) NHS, and let the material crosslink for 2 h at 25 degrees Celsius. Rinsing the gel 3 times in PBS, with final wash of 12 hours in PBS. Pipetting out the PBS and seeding CECs on the hydrogel surface.

### Example 2

Spreading 100 μL of 5% (w/v) gelatin methacrylate on an area of 10 mm×10 mm, on a flat hydrophobic plastic surface (e.g. Petri dish), to form a layer of about 1.2 mm thick. Letting the material dry in a 25 degrees Celsius vacuum desiccator for 12 hours. Adding 2 ml PBS solution with (0.5% w/v) LAP, and applying UV light at 1 mW/cm\hat{ }2 to crosslink the material for 30 minutes. Rinsing the gel 3 times in PBS, with final wash of 12 hours in PBS. Pipetting out the PBS and seeding CECs on the hydrogel surface.

It is according to a few embodiments of the invention wherein grafts and implanted via a few optional procedures, see examples 3-4.

### Example 3

The artificial EK graft is implanted using standard DMEK or DSEK techniques and tools, such as Coronet Endoglide (commercially available by Network Medical Products) or Geuder cannula. This includes loading the graft onto the tool (before or after shipment), inserting the tool through a peripheral corneal incision, injecting/pulling/pushing the graft into the patient's anterior chamber, and attaching the graft to the posterior cornea with an air bubble.

### Example 4

To make the implantation procedure easier and safer, a designated carrier device is used to insert the graft into the patient's eye (see FIG. 2). The carrier is made of detachable/dissolvable thermo-responsive materials, with a diameter which is similar to the graft or slightly larger. The EK graft is attached to the carrier device, folded and inserted into the eye. After insertion, an air bubble is injected to attach the graft to the posterior cornea. Then, the cornea is warmed to 34-38 degrees Celsius to detach or dissolve the carrier.

All references cited throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, and method steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and sub-combinations possible of the group are intended to be individually included in the disclosure. When a compound is described herein such that a particular isomer, enantiomer or diastereomer of the compound is not specified, for example, in a formula or in a chemical name, that description is intended to include each isomer and enantiomer of the compound described individually or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are described. Nothing herein is to be construed as an admission that the invention is not entitled to antedate such disclosure by virtue of prior invention.

Whenever a range is given in the specification, for example, a range of integers, a temperature range, a time range, a composition range, or concentration range, all intermediate ranges and subranges, as well as all individual values included in the ranges given are intended to be included in the disclosure. As used herein, ranges specifically include the values provided as endpoint values of the range. As used herein, ranges specifically include all the integer values of the range. For example, a range of 1 to 100 specifically includes the end point values of 1 and 100. It will be understood that any subranges or individual values in a range or subrange that are included in the description herein can be excluded from the claims herein. The term “about” refers to any value being lower or greater than 20% of the defined measure.

As used herein, “comprising” is synonymous and can be used interchangeably with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” can be replaced with either of the other two terms. The invention illustratively described herein suitably can be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments and optional features, modification and variation of the concepts herein disclosed can be resorted to by those skilled in the art, and that such modifications and variations are considered to be within the scope of this invention as defined by the appended claims.

1. An artificial endothelial keratoplasty graft consisting a support layer made of rehydrated crosslinked hydrogel and corneal endothelial cells on top or within said support layer.

2. The graft of claim 1, wherein the rehydrated crosslinked hydrogel is based on crosslinked collagen or collagen methacrylate.

3. The graft of claim 1, wherein the source of said collagen or collagen methacrylate is human recombinant collagen.

4. The graft of claim 1, wherein the rehydrated crosslinked hydrogel is based on crosslinked gelatin or gelatin methacrylate.

5. The graft of claim 1, wherein the total thickness of the graft is between 5 and 50 microns.

6. The graft of claim 1, wherein the total thickness of the graft is below 25 microns, therefore mimicking native DMEK graft.

7. The graft of claim 1, wherein the mechanical properties of said support layer allows positioning and flattening of the graft inside the anterior chamber during implantation.

8. The graft of claim 1, wherein said cells remodeled the said support layers to form corneal endothelium extra-cellular matrix, by a long maturation period or by additional nutritions to the cells.

9. A method of manufacturing an artificial endothelial keratoplasty graft, wherein said method consisting of a step of drying support layer material followed by a crosslinking step.

10. The method of claim 9, wherein the support layer material is collagen or collagen methacrylate.

11. The method of claim 9, wherein the source of said collagen or collagen methacrylate is human recombinant collagen.

12. The method of claim 9, wherein the support layer material is gelatin or gelatin methacrylate.

13. The method of claim 9, wherein the crosslinking step involves in introducing EDC and NHS molecules to the material.

14. The method of claim 9, wherein said crosslinking step involves in introducing LAP or Irgacure 2959 molecules to the material and applying light on it.

15. The method of claim 9, wherein said drying step is performed in controlled environmental conditions.

16. The method of claim 9, wherein the final thickness of said graft is below 25 microns.

17. The method of claim 9, wherein the mechanical properties of said graft allow pulling and pushing it during implantation, therefore allowing positioning and flattening inside the anterior chamber of the eye.

18. The method of claim 9, wherein the water content in said support material is between 30 and 90 percent.
